(19) United States
(12) Patent Application Publication
(10) Pub. No.: US 2008/0312630 A1
(43) Pub. Date: Dec. 18, 2008

(54) Sanitary Product

(76) Inventor: Jung Kyung Seo, New South Wales (AU)

Correspondence Address: STETINA BRUNDA GARRED & BRUCKER, 75 ENTERPRISE, SUITE 250, ALISO VIEJO, CA, 92656, US

(21) Appl. No.: 11/884,180

(22) PCT Filed: Feb. 1, 2006
(86) PCT No.: PCT/AU06/00110

§ 371 Date: Feb. 28, 2008

(30) Foreign Application Priority Data
Feb. 11, 2005 (AU) 2005900657
May 31, 2005 (AU) 2005100460

(51) Int. Cl. A61F 13/15

(52) U.S. Cl. 604/385.03; 604/385.01; 604/389

(57) ABSTRACT

A sanitary pad auxiliary appendage (10) for separate attachment at the rear end (38) of a sanitary pad (100) so as to form an extension of said sanitary pad, said auxiliary appendage comprising a base portion (12) and a fin-like structure (14) extending at least a portion of the length of said base portion, said fin-like structure projecting upwardly from said base portion, said fin-like structure adapted to at least partially fill a crevice between the buttocks of a user so as to form a barrier between the thighs and buttocks of said user.

The present invention relates to sanitary products for women and, more particularly to sanitary pads for the control of menstrual flows.

### BACKGROUND

Sanitary pads for control and absorption of the monthly discharge of blood and mucosal tissue from the vagina have a long evolutionary history from simple cloths to the sophisticated sanitary pads available today.

Nevertheless, for many women the use of available pads is still accompanied by anxiety over their effectiveness, particularly so in some particular use contexts. Thus many women will avoid sleeping lying supine, or will do so worrying about the possibility of leakage.

Nor is this cause for anxiety restricted to sleeping in this position. Even sitting in a more relaxed or reclined posture may, especially for those women suffering a heightened degree of discharge, raise the fear of marked clothing and embarrassment.

Leakage past the rear end of conventional pads when the body is in one of the above described positions, is possible because the essentially flat pads cannot conform closely enough to the junction between the perineum, thighs and buttocks, to prevent passage of the menses through the crevice formed between them.

“Overnight” sanitary pads are known but these only provide additional length flat pad and cannot prevent seepage through the crevice between the buttocks.

Also known in the prior art are sanitary pads which incorporate at their rearward end some form of raised structure for ameliorating rearward flow of the menses. These pads however are difficult to position and the projections provided generally inadequate.

It is an object of the present invention to address or ameliorate some of the above disadvantages.

### BRIEF DESCRIPTION OF INVENTION

Accordingly, in a first broad form of the invention, there is provided a sanitary pad auxiliary appendage for separate attachment at the rear end of a sanitary pad so as to form an extension of said sanitary pad; said auxiliary appendage comprising a base portion and a fin-like structure extending at least a portion of the length of said base portion; said fin-like structure projecting upwardly from said base portion; said fin-like structure adapted to at least partially filling the crevice between the buttocks of a user so as to form a barrier between the thighs and said buttocks of said user by contact between said fin-like structure and at least a portion of the perineum or the furrow formed at the junction of the buttocks between vagina and coccyx, thereby to prevent seepage of bodily fluids from a rear end of said sanitary pad.

Preferably, said fin-like structure extends rearwardly from proximate a forward edge of said base portion.

Preferably, said fin-like structure comprises an absorbent, elongate flexible and resilient structure; said structure formed of side portions and a first leading end and a second trailing end.

Preferably, at least portions of said side portions and said second trailing end are impervious.

Preferably, at least portions of said side portions and said first leading end are of a permeable fabric or another suitable moisture absorbing material.

Preferably, said side portions, said first leading end and said second trailing end enclose a wadding of absorbent material.

Preferably, a forward section of said base portion is provided with at least one adhesive area on at least one surface of said forward section; said one or more adhesive areas protected prior to use by a peel-off covering strip; said at least one adhesive area adapted to attach said auxiliary appendage to said rear end of said sanitary pad.

Preferably, said at least one adhesive area of said forward section is provided at an upper surface of said forward section; said at least one adhesive area adapted to attach said appendage to an underside of said rear end of said sanitary napkin.

Preferably, said at lest one adhesive area of said forward section are provided at an underside of said forward section; said at least one adhesive area adapted to attach said appendage to an upper surface of said rear end of said sanitary napkin.

Preferably, at least a border at the periphery of said underside of said base portion is provided with an adhesive layer; said layer protected prior to use by a peel-off layer; said adhesive layer adapted for releasably attaching said appendage to an inside surface of an article of underwear.

Preferably, a border at the periphery of said upper surface of said base portion is provided with an adhesive layer; said adhesive layer protected prior to use by a peel-off layer of soft flexible resilient and non-irritating material; said adhesive layer adapted to releasably attach said base portion to the skin of a user.

Preferably, height, length ad thickness of said fin-like structure are provided in a plurality of sizes to suit selected ranges of body sizes of users.

Preferably, said fin-like structure is provided in a selection of grades of absorbency to suit variations in volume of said menstrual discharge or bodily fluids.

Preferably, said fin-like structure is provided with a base portion prepared with an adhesive; said fin-like structure adapted to be adhesively applied to an upper surface of said base portion prior to use.

Preferably, said adhesive is protected by a peel-off layer prior to use.

Preferably, said fin-like structure extends upwardly from a substantially flat said base portion.

Preferably, said fin-like structure extends upwardly from a curved base portion; said base portion curved upwardly to substantially conform to curvature of said buttocks of said user.

Preferably, said base portion is substantially of rectangular form.

Preferably, said base portion widens outwardly and rearwardly from said first leading end.

Preferably, said base portion extends rearwardly beyond said second trailing end of said fin-like structure.

Preferably, at least a portion of an upper surface of said base portion is provided with absorbent padding.

In a further broad form of the invention, there is provided a method for preventing leakage of menstrual flow from a rear end of a sanitary pad; said method including the steps of:
(a) combining said sanitary pad with an auxiliary appendage comprising a base portion and an upwardly extending fin-like structure,
(b) applying said auxiliary appendage such that said fin-like structure forms a barrier between the buttocks and thighs of a user; said barrier in contact with some portion of the perineal area or its extension rearward towards the coccyx.

Preferably, said fin-like structure is integral with said base portion.

Preferably, said auxiliary appendage is applied adhesively to said rear end of said sanitary pad prior to application to the body of said user.

Preferably, said auxiliary appendage is applied adhesively to said body of said user.

Preferably, said auxiliary appendage is applied adhesively to the underwear of a user.

### BRIEF DESCRIPTION OF DRAWINGS

Embodiments of the present invention will now be described with reference to the accompanying drawings wherein:

FIG. 1 is a perspective view of a sanitary product according to a preferred embodiment of the present invention,

FIG. 2 is a side elevation view of the sanitary product of FIG. 1,

FIG. 3 is a partial sectioned side view of the female lower form with a sanitary pad and the sanitary product of FIGS. 1 and 2 in use,

FIG. 4 is a rear view of part of a user with a preferred form of the sanitary product of FIGS. 1 to 3 in use,

FIG. 5 is a perspective view of a second preferred embodiment of a sanitary product according to the invention,

FIG. 6 is a perspective view of a third preferred embodiment of a sanitary product,

FIG. 7 is a side view of the embodiment shown in FIG. 6,

FIG. 8 is a perspective view of a fourth preferred embodiment of a sanitary product,

FIG. 9 is a side view of the embodiment of FIG. 8 indicating alternative arrangements of attachment to a conventional sanitary pad,

FIG. 10 is a side view of a sanitary product according to the invention showing one preferred configuration of a fin-like structure,

FIG. 11 is a view of the underside of one embodiment of the sanitary product of the invention with a preferred arrangement of adhesive areas,

FIG. 12 is a further side view of a sanitary product with a further preferred configuration of a fin-like structure.

### DETAILED DESCRIPTION OF PREFERRED EMBODIMENTS

The problem of seepage of menstrual discharge or other bodily fluids from the rear end of a conventionally constructed, substantially flat sanitary pad, is experienced by many women and particularly by those women suffering a heavier than average discharge.

The rear end of a flat sanitary pad when in use, generally locates in the region of the lower portion of the buttocks. Especially when a wearer is in a supine position, or even when sitting in a relaxed position, seepage of the menses may pass into the crevice between the buttocks and escape past the rear end of the pad.

The present invention provides an auxiliary appendage, which may be attached to the rear of a conventional flat sanitary pad, particularly in situations where the discharge is heavy and for use at night. FIG. 1 shows an auxiliary appendage 10 attached to the rear end 38 of a conventional flat sanitary pad 100. As well as being attachable to a conventional sanitary pad, the present invention also encompasses alternative arrangements in which the auxiliary pad is attached either to the underwear or the skin of a user.

### First Preferred Embodiment

With reference to FIG. 1 a base portion 12 of an auxiliary appendage 10 is constructed in the manner of a conventional sanitary pad for the absorption of menstrual discharge. Auxiliary appendage 10 is provided with a fin-like structure 14 projecting upwardly from the upper surface 16 of base portion 12.

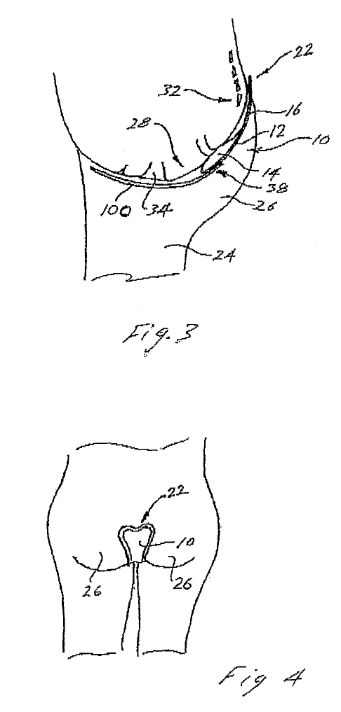

As may be seen in FIGS. 1 and 3, the fin-like structure 14 of auxiliary appendage 10 is adapted for insertion between the thighs 24 and buttocks 26 of a user so as to contact at least a portion of the perineal region 28 or its extension rearward of the vagina 34 and extending towards the coccyx 32. Thus in use, as can be seen in FIG. 3 the rear edge 22 of base portion 12 may be positioned considerably further towards the coccyx than is possible with conventional napkins. Indeed, the outer end of the base portion may project from between the buttocks 26, covering the region of the coccyx, and in at least some preferred forms, base portion 12 may be given a decorative shape, for example heart shaped as shown in FIG. 4.

Base portion 12 is constructed of a supporting layer of material overlaid with soft absorbent padding layers, both being of similar material and consistency as commonly used in the construction of sanitary pads.

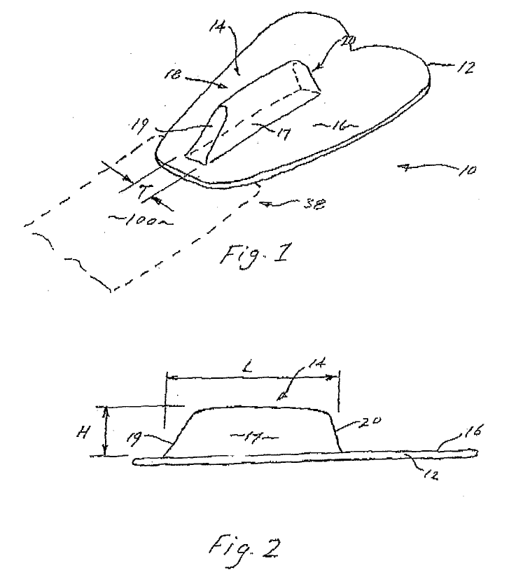

With reference now to FIGS. 1 and 2, fin-like structure 14 of auxiliary appendage 10 is comprised of side portions 17 and 18 respectively, first leading end portion 19 and second trailing end portion 20. These side portions and end portions form an envelope for an absorbent wadding (not shown) so that the fin-like structure 14 is both flexible and resilient.

Preferably, though not essentially, the lower parts of each of side portions 17 and 18 and all of trailing end portion 20 are of an impervious material, with the remaining areas of side portions 17 and 18 and leading end portion 19 permeable.

Auxiliary appendage 10 and its fin-like structure 14 may be provided in a range of sizes to suit variations in the body sizes of users and variations in volume of menstrual discharge. Thus the fin-like structure may be made available in a range of heights “H”, lengths “L” and thicknesses “T”, as shown in FIGS. 1 and 2. As well, the fin-like structure 14 and the base portion 12 may be provided in selected grades of absorbency. The shape of fin-like structure 14 may also be made available in different configurations to suit the preferences of individual users. Examples of possible preferred shapes are shown in FIGS. 10 and 12.

By being wedged between the thighs 24 and buttocks 26 and in contact with at least a portion of the perineal area 28 or its extension rearward of the vagina 34 towards the coccyx 32, auxiliary appendage 10 acts as a barrier to fluids leaking towards the back of the body, particularly when the body is supine for sleep or even when in a reclined sitting position.

### Second Preferred Embodiment

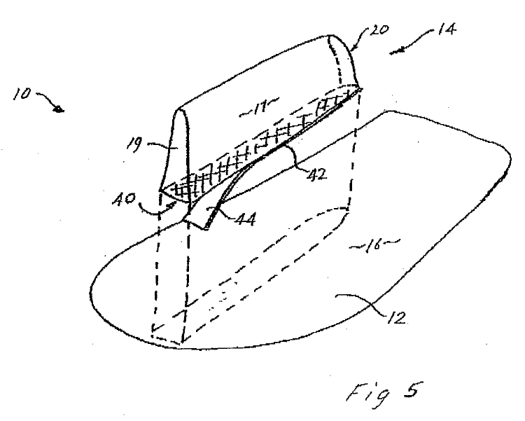

With reference to FIG. 5, in a second preferred embodiment Of the invention, fin-like structure 14 is provided separately from base portion 12. In addition to the side portions 17 and 18, leading end portion 19 and trailing end portion 20 already described above, fin-like structure 14 of this embodiment is provided with a generally flat underside 40. Underside 40 is prepared with an adhesive layer 42, protected prior to use by a peel-off strip 44.

Base portion 12 may thus be prepared as required, for example for night use, by removing strip 44 from underside 40 of fin-like structure 14 and applying it to the base portion 12. Adhesive layer 42 is formulated to be of sufficient strength so that once applied, fin-like structure 14 cannot accidentally be dislodged from the base portion 12. In this embodiment, base portions 12 and fin-like structures 14 may be provided in various sizes and absorbencies to suit an individual's requirements.

### Third Preferred Embodiment

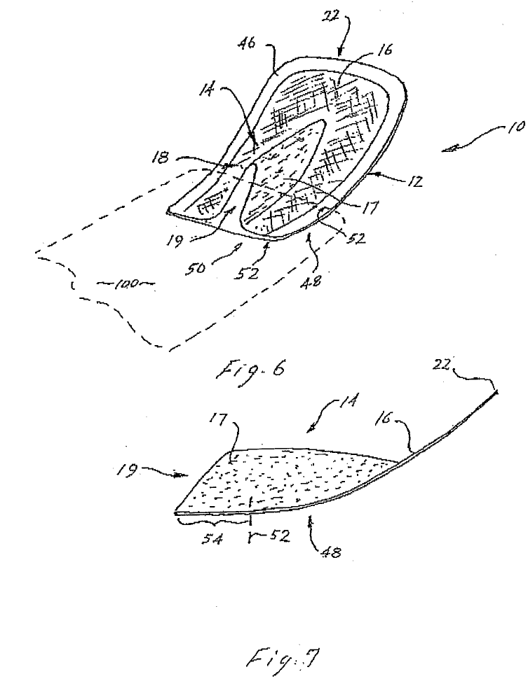

In a third preferred embodiment of the invention, fin-like structure 14 may be formed of the same absorbent padding layer material as base portion 12, and may be integral with it, or may be separately constructed but permanently affixed to base portion 12. With reference to FIG. 6, fin-like structure 14 is positioned proximate the forward edge 50 of base portion 12. Base portion 12 is preferably somewhat curved, as best seen in FIG. 7, so as to conform more readily to the contour of the buttocks 26.

The auxiliary appendage 10 of this embodiment is also adapted for attachment at the rear of a conventional sanitary pad 100 as indicated in FIG. 1 to form an extension of the pad.

With reference to FIGS. 6 and 7, fin-like structure 14 is again comprised of side portions 17 and 18 respectively and leading end portion 19. However, in this embodiment fin-like structure 14 extends rearwardly to meld with the upper surface 16 of curved base portion 12.

The underside 48 of base portion 12 is provided with at least an adhesive portion 54, forward of dashed line 52, in the region intended for adhesion to a sanitary pad 100.

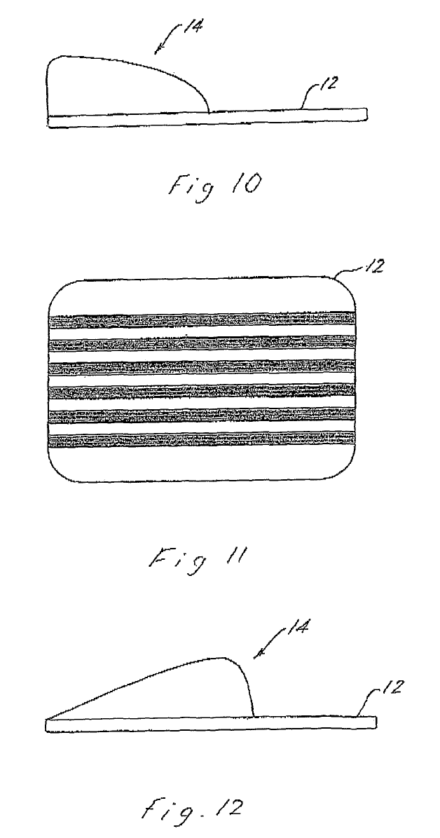

The adhesive region 54 forward of dashed line 52 is protected prior to use by a peel-off layer (not shown). In at least one form of this embodiment, an adhesive portion may form at least a border extending along the complete periphery of the underside 48 of base portion 12 with protective stripping divided at dashed line 52. Alternatively, the whole of underside 48 may be provided with an adhesive layer, or the underside may be provided with a number of adhesive areas, for example in strips as shown in FIG. 11. As well as providing for adhesion to a sanitary pad 100, this arrangement allows for the auxiliary appendage to also be adhered to the inside of the underwear of a user.

Again with reference to FIG. 6, in at least one further preferred form of this embodiment, the periphery of base portion 12 is provided on the upper surface 16 with an adhesive border 46 allowing a user to adhere the auxiliary appendage 10 to the skin. The peel-off layer protecting any adhesive layer in this embodiment is made of a soft non-irritating material so that where an adhesive layer is not required and the protective layer remains in place, it does not cause irritation to the skin of a user.

### Fourth Preferred Embodiment

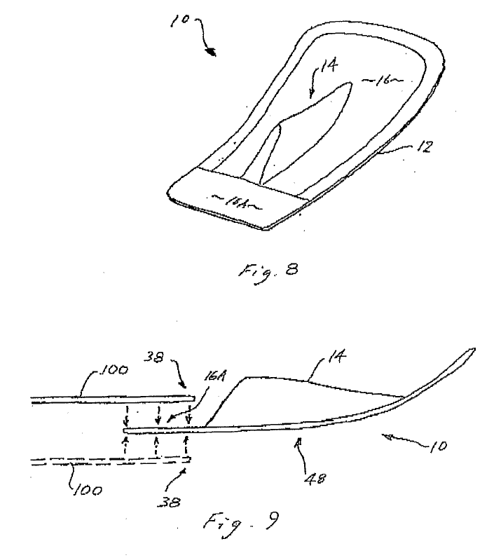

Alternatively, with reference to FIGS. 8 and 9 auxiliary appendage 10 may be adapted for attachment to a conventional flat sanitary pad 100 by adhesion either to the upper surface or to the underside of the sanitary pad 100. To enable the auxiliary appendage 10 to be attached to the underside of the rear end 38 of a conventional sanitary pad 100, base portion 12 is extended somewhat forwardly of fin-like structure 14 as shown in FIG. 8 to provide an adhesion area 16A. Adhesion area 16A is provided with a protective cover strip prior to use (not shown).

### In Use

In use, the configuration of the auxiliary appendage of the present invention allows for ease of positioning the appendage for protection against rearward leakage. Because it is specifically designed for relatively deep insertion between the buttocks, it is retained in that position with little risk of slippage while putting on underwear. Where desired, the adhesive facility of the appendage allows accurate positioning and attachment to a user's underwear. The adhesive connection between the appendage and the rear end of a conventional sanitary pad ensures that any fluid passing that rear end is safely transferred to the auxiliary appendage and absorbed by the blocking action of the fin-like structure.

It will be understood that the auxiliary appendage may be applied for use in a number of ways. The appendage may firstly be applied directly to the body of a user, after which a conventional sanitary pad is applied so that its rear end extends under the forward portion of the appendage to attach to the adhesive section. Or, prior to application, a user may attach the auxiliary appendage to a sanitary pad by means of the adhesive provided on the appendage and apply the combined sanitary pad and appendage to the body or to the underwear. Or yet again, the auxiliary appendage, either already combined with a sanitary pad or by itself, may be affixed inside underwear by means of the adhesive on the underside of the appendage.

The above describes only some embodiments of the present invention and modifications, obvious to those skilled in the art, can be made thereto without departing from the scope and spirit of the present invention. Thus various modifications may be made to the arrangement of adhesive strips and layers and to the shape of the base portion for example.

1. A sanitary pad auxiliary appendage for separate attachment at the rear end of a sanitary pad so as to form an extension of said sanitary pad, said auxiliary appendage comprising a base portion and a fin-like structure extending at least a portion of the length of said base portion, said fin-like structure projecting upwardly from said base portion, said fin-like structure adapted to at least partially filling the crevice between the buttocks of a user so as to form a barrier between the thighs and said buttocks of said user by contact between said fin-like structure and at least a portion of the perineum or the furrow formed at the junction of the buttocks between vagina and coccyx, thereby to prevent seepage of bodily fluids from a rear end of said sanitary pad.

2. The appendage of claim 1, wherein said fin-like structure extends rearwardly from proximate a forward edge of said base portion.

3. The appendage of claim 1, wherein said fin-like structure comprises an absorbent, elongate flexible and resilient structure, and said structure is formed of side portions and a first leading end and a second trailing end.

4. The appendage of claim 3, wherein at least portions of said side portions and said second trailing end are impervious.

5. The appendage of claim 3, 4 wherein at least portions of said side portions and said first leading end are of a permeable fabric or another suitable moisture absorbing material.

6. The appendage of claim 3, wherein said side portions, said first leading end and said second trailing end enclose a wadding of absorbent material.

7. The auxiliary appendage of claim 1, wherein a forward section of said base portion is provided with at least one adhesive area on at least one surface of said forward section, said one or more adhesive areas are protected prior to use by a peel-off covering strip; and said at least one adhesive area is adapted to attach said auxiliary appendage to said rear end of said sanitary pad.

8. The auxiliary appendage of claim 7, wherein said at least one adhesive area of said forward section is provided at an upper surface of said forward section, and said at least one adhesive area is adapted to attach said appendage to an underside of said rear end of said sanitary napkin.

9. The auxiliary appendage of claim 7, wherein said at lest one adhesive area of said forward section are provided at an underside of said forward section, and said at least one adhesive area is adapted to attach said appendage to an upper surface of said rear end of said sanitary napkin.

10. The appendage of claim 8, wherein at least a border at the periphery of said underside of said base portion is provided with an adhesive layer, said layer is protected prior to use by a peel-off layer; and said adhesive layer is adapted for releasably attaching said appendage to an inside surface of an article of underwear.

11. The appendage of claim 8, wherein a border at the periphery of said upper surface of said base portion is provided with an adhesive layer, said adhesive layer protected prior to use by a peel-off layer of soft flexible resilient and non-irritating material, and said adhesive layer is adapted to releasably attach said base portion to the skin of a user.

12. The appendage of claim 1, wherein height, length ad thickness of said fin-like structure are provided in a plurality of sizes to suit selected ranges of body sizes of users.

13. The appendage of claim 1, wherein said fin-like structure is provided in a selection of grades of absorbency to suit variations in volume of said menstrual discharge or bodily fluids.

14. The appendage of claim 1, wherein said fin-like structure is provided with a base portion prepared with an adhesive, and said fin-like structure is adapted to be adhesively applied to an upper surface of said base portion prior to use.

15. The appendage of claim 14 wherein said adhesive is protected by a peel-off layer prior to use.

16. The appendage of claim 1, wherein said fin-like structure extends upwardly from a substantially flat said base portion.

17. The appendage of claim 1, wherein said fin-like structure extends upwardly from a curved base portion, and said base potion is curved upwardly to substantially conform to curvature of said buttocks of said user.

18. The appendage of claim 1, wherein said base portion is substantially of rectangular form.

19. The appendage of claim 1, wherein said base portion widens outwardly and rearwardly from said first leading end.

20. The appendage of claim 3, wherein said base portion extends rearwardly beyond said second trailing end of said fin-like structure.

21. The appendage of claim 1, wherein at least a portion of an upper surface of said base portion is provided with absorbent padding.

22. A method for preventing leakage of menstrual flow from a rear end of a sanitary pad; said method including the steps of:
(a) combining said sanitary pad with an auxiliary appendage comprising a base portion and an upwardly extending fin-like structure,
(b) applying said auxiliary appendage such that said fin-like structure forms a barrier between the buttocks and thighs of a user; said barrier in contact with some portion of the perineal area or its extension rearward towards the coccyx.

23. The method of claim 22 wherein said fin-like structure is integral with said base portion.

24. The method of claim 22, wherein said auxiliary appendage is applied adhesively to said rear end of said sanitary pad prior to application to the body of said user.

25. The method of claim 22, wherein said auxiliary appendage is applied adhesively to said body of said user.

26. The method of claim 22, wherein said auxiliary appendage is applied adhesively to the underwear of a user.

27. (canceled)
